(19) United States
(12) Patent Application Publication
(10) Pub. No.: US 2008/0166297 A1
(43) Pub. Date: Jul. 10, 2008

(54) Radionuclide-Chitosan Complex Having an Improved Stablilized Gelatin in Administering Them to the Body and Their Preparation Method

(75) Inventors: Jei-Man Ryu, Kyunggi-do (KR); Dong-Hyuk Shin, Kyunggi-do (KR); Seung-Kyoo Seong, Kyunggi-do (KR); Hee-Bog Yang, Kyunggi-do (KR); Young-Jun Song, Seoul (KR); Eun-Jung Bae, Seoul (KR); Kyung-Bae Park, Taejeon-si (KR); Byung-Chul Shin, Taejeon-si (KR)

Correspondence Address: LUCAS & MERCANTI, LLP, 475 PARK AVENUE SOUTH, 15TH FLOOR, NEW YORK, NY, 10016, US

(73) Assignees: DONG WHA PHARM. IND. CO., LTD., Seoul (KR); KOREAN ATOMIC ENERGY RESEARCH INSTITUTE, Taejeon-si (KR)

(21) Appl. No.: 11/720,572

(22) PCT Filed: Dec. 1, 2005
(86) PCT No.: PCT/KR05/04083

§ 371 Date: May 31, 2007

(30) Foreign Application Priority Data
Dec. 1, 2004 (KR) 10-2004-0099798

(51) Int. Cl. A61K 51/04; C07F 5/00

(52) U.S. Cl. 424/1.73; 534/10; 534/11

(57) ABSTRACT

The present invention relates to a radionuclide-chitosan complex solution and its preparation method, and more particularly to the radionuclide-chitosan complex solution having a viscosity of 300˜2,400 cps, comprising an aqueous chitosan solution or a freeze-dried chitosan labeled with a radionuclide. The radionuclide-chitosan complex solution according to the present invention has a stable gelation state at a target region when injected into the body while maintaining a labeling yield of radioisotope to chitosan above 99%. Side effects may be minimized and treatment efficiency may be increased when injected to a patient.

### TECHNICAL FIELD

The present invention relates to a radionuclide-chitosan complex solution that almost never leaks radioactive materials into other regions out of the target region by gelation in the body while the labeling yield of a radioisotope to chitosan is maintained above 99%, and its preparation method.

### BACKGROUND ART

Korean Patent No. 190,957 discloses a complex of a chitosan and a radionuclide emitting high energy β-ray and low energy γ-ray at the same time (hereinafter, referred to as “radionuclide-chitosan complex”). One of the most important properties of the radionuclide-chitosan complex solution is that transcutaneous injection is possible because the complex solution exists as a liquid in a weakly acidic condition. The complex solution transcutaneously injected at a region as a liquid is neutralized with body fluid and then is gelated. The injected complex solution is retained at an injected region and shows a medical treatment effect. Therefore, an internal radioactive treatment is possible. The viscosity of a chitosan solution disclosed in Korean Patent No. 190,957 should preferably be 100˜200 cps for the preparation of a radionuclide-chitosan complex solution in order to obtain a labeling yield above 99%.

However, in the case of a radionuclide-chitosan complex solution, not only a labeling yield but also retaining, through gelation, at an injected region without diffusing radioactive materials into other regions are very important for stability. That is, a gelation state of a radionuclide-chitosan complex solution is very important for preventing an injected radionuclide-chitosan complex solution from diffusing into other regions. It is preferable that gelation should occur without dispersion just after injection of a complex solution into the body. There is a direct relationship between a gelation state and a viscosity of a radionuclide-chitosan complex solution.

The inventors found that a radionuclide-chitosan complex solution prepared by using a chitosan solution having a viscosity of 100˜200 cps described in Korean Patent No. 190,957 showed a labeling yield above 99% and is suitable for injection. However, the radionuclide-chitosan complex solution was not Good in gelation but the gel was dispersed in the body. Therefore they found that there were many chances of leakage of radioactive materials into other regions out of the target region.

TABLE 1

| | 1 | 2 |
|---|---|---|
| Viscosity of chitosan | 146 | 376 |
| solution | | |
| Viscosity of complex | 107 | 281 |
| solution | | |
| Gelation state | Most gels dispersed | Gels partially |
| | | dispersed |
| Remarks | Easy for injection | Easy for injection |

According to the above result, for a gelation stability of a radionuclide-chitosan complex solution in the body condition, a radionuclide-chitosan complex solution having a much higher viscosity is required than a radionuclide-chitosan complex solution prepared by using a chitosan solution having a viscosity of 100˜200 cps described in Korean Patent No. 190,957.

As described in Korean Patent No. 190,957, the labeling yield is above 99% when the viscosity of a chitosan solution is above 100 cps. Therefore, the labeling yield of the radionuclide-chitosan complex according to the present invention is above 99% because the viscosity of a chitosan solution required in the present invention is higher than the viscosity described in Korean Patent No. 190,957.

The inventors of the present invention completed a radionuclide-chitosan complex solution having a labeling yield above 99% whose radioactive materials are almost never leaked into neighboring regions by studying viscosities of radionuclide-chitosan complex solutions.

### DISCLOSURE OF THE INVENTION

### Technical Problem

An object of the present invention is to provide a radionuclide-chitosan complex solution that almost never leaks radioactive materials into other regions out of a target region by gelation after injection into the body while maintaining a labeling yield above 99%, and its preparation method.

### Technical Solution

To accomplish the object, the present invention provides a radionuclide-chitosan complex solution having a viscosity of 300˜2,400 cps prepared by adding a radionuclide solution into a chitosan having a molecular weight of 460,000˜1,570,000 dissolved in an aqueous weak acid solution, and its preparation method.

### Advantageous Effect

A radionuclide-chitosan complex solution according to the present invention has advantages that the labeling yield above 99% is maintained and radioactive materials are almost never leaked into other regions out of a target region by maintaining a stable gelation of the radionuclide-chitosan complex solution at a target region when injected into the body.

### BRIEF DESCRIPTION OF THE DRAWINGS

FIGS. 1a and 1b are pictures showing a gelation state and dispersed gels of a holmium-chitosan complex solution having a viscosity of 60 cps in accordance with a comparative example of the present invention.

FIG. 2 shows pictures of gelation states of a holmium-chitosan complex solution having a viscosity of 117 cps in accordance with a comparative example of the present invention.

FIG. 3 shows pictures of gelation states of a holmium-chitosan complex solution having a viscosity of 194 cps in accordance with a comparative example of the present invention.

FIG. 4 is a picture showing a gelation state of a holmium-chitosan complex solution having a viscosity of 289 cps in accordance with a comparative example of the present invention.

FIG. 5 is a picture showing a gelation state of a holmium-chitosan complex solution having a viscosity of 310 cps in accordance with an example embodiment of the present invention.

FIG. 6 is a picture showing a gelation state of a holmium-chitosan complex solution having a viscosity of 650 cps in accordance with an example embodiment of the present invention.

FIG. 7 is a picture showing a gelation state of a holmium-chitosan complex solution having a viscosity of 1,068 cps in accordance with an example embodiment of the present invention.

FIG. 8 is a picture showing a gelation state of a holmium-chitosan complex solution having a viscosity of 1,407 cps in accordance with an example embodiment of the present invention.

FIG. 9 is a picture showing a gelation state of a holmium-chitosan complex solution having a viscosity of 2,376 cps in accordance with an example embodiment of the present invention.

FIG. 10 is a picture showing a gelation state of a holmium-chitosan complex solution having a viscosity of 2,549 cps in accordance with a comparative example of the present invention.

FIGS. 11a and 11b are pictures showing a dispersed state of a holmium-chitosan complex solution in accordance with a comparative example of the present invention and a gelation state in accordance with an example embodiment of the present invention.

### BEST MODE

To accomplish object, the present invention provides a radionuclide-chitosan complex solution having a viscosity of 300˜2,400 cps prepared by adding a radionuclide solution (Kit A) into a freeze-dried chitosan (Kit B) prepared by dissolving a chitosan having a molecular weight of 460,000˜1,570,000 in an aqueous weak acid solution and freeze-drying.

To accomplish another object, the present invention provides a preparation method of the radionuclide-chitosan complex solution having a viscosity of 300˜2,400 cps, comprising the steps of

(1) preparing an aqueous radionuclide solution by irradiating a stable nuclide selected from the group consisting of ¹⁶⁴Dy(NO₃)₃, ¹⁶⁴Dy₂O₃, ¹⁶⁵Ho(NO₃)₃ and ¹⁶⁵Ho₂O₃ with neutrons in a nuclear reactor to transform into a radionuclide and dissolving the radionuclide in distilled water;

(2) preparing a chitosan solution having a viscosity of 380˜2,500 cps by dissolving a chitosan having a molecular weight of 460,000˜1,570,000 in a weak acid solution; and

(3) adding the radionuclide solution prepared in step (1) into the chitosan solution prepared in step (2).

In a method for the preparation of a radionuclide-chitosan complex solution in accordance with the present invention, step (2) may further comprise a step of preparing a freeze-dried chitosan by freeze-drying the prepared chitosan solution. In this case, the chitosan solution of step (3) is a freeze-dried chitosan.

In addition, the present invention provides a method for treating liver cancer by using the radionuclide-chitosan complex solution having a viscosity of 300˜2,400 cps.

According to the radionuclide-chitosan complex solution of the present invention, side effects can be minimized when injected into a patient and excellent treatment efficiency is expected for the treatment of a cystic cancer such as liver cancer, because the labeling yield of a radioactive isotope to chitosan is maintained above 99% and a stable gelation is maintained in the target region when injected to the body.

Hereinafter, the present invention will be described in more detail.

A gelation state of a radionuclide-chitosan complex solution in the body is determined by a viscosity of the complex solution. In addition, the viscosity of a radionuclide-chitosan complex solution is proportional to the molecular weight of a chitosan. Accordingly, chitosans having various molecular weights are used to prepare the radionuclide-chitosan complexes that almost never leak radioactive materials into other regions out of the target region through gelation in the body when injected into the body.

In addition, a molecular weight of the chitosan, viscosity of the chitosan solution, and viscosity of the complex solution are determined by confirming gelation states of the complex solutions prepared in a buffer solution having a pH similar to the internal condition of the body.

Firstly, a radionuclide-chitosan complex solution according to the present invention will be described.

The radionuclide-chitosan complex solution according to the present invention may be prepared by adding a radionuclide solution into a chitosan solution. The chitosan solution may be prepared by dissolving a chitosan in a weak acid solution.

The molecular weight of a chitosan is preferably 460,000˜1,570,000. When the molecular weight of a chitosan is less than 460,000, the viscosity of the prepared complex solution is so low that gel is dispersed when injected into the body, and radioactive materials may be leaked into other regions out of a target region. When the molecular weight of a chitosan is more than 1,570,000, the viscosity of the prepared complex solution is so high that injection is difficult.

As the weak acid, any weak acid may be used, carboxylic acid such as formic acid and acetic acid may preferably be used, and acetic acid is more preferably used.

In an example embodiment according to the present invention, 20 mg of chitosan having a molecular weight of 460,000˜1,570,000 is dissolved in 2 mL of 1% acetic acid solution to obtain a chitosan solution having a viscosity of 380˜2,500 cps. When the viscosity of the chitosan solution is lower than 380 cps, the viscosity of a prepared complex solution is so low that gels are dispersed when injected into the body, and radioactive materials may be leaked into other regions out of a target region. When the viscosity of a chitosan solution is higher than 2,500 cps, the viscosity of a prepared complex solution is so high that injection is difficult.

And, a radionuclide solution is prepared by irradiating stable nuclides with neutrons in a nuclear reactor and then dissolving in distilled water. The stable nuclide compound may be an oxide or nitrate of ¹⁶⁵Ho or ¹⁶⁴Dy, and more preferably an oxide or nitrate of ¹⁶⁵Ho.

In an example embodiment according to the present invention, 10% aqueous radionuclide solution of ¹⁶⁶Ho(NO₃)₃19.5H₂O is prepared by irradiating 200 mg of ¹⁶⁵Ho(NO₃)₃19.5H₂O for 50 hours in an irradiation hole of a nuclear reactor(Hanaro reactor at Korea Atomic Energy Research Institute) where the velocity of thermal neutron is 4.0×10¹³n/cm²·sec. and dissolving the radionuclides in distilled water.

Subsequently the present invention provides kits for preparing a radionuclide-chitosan complex solution. The kits are Kit A of an aqueous solution of a radioactive material and Kit B of a chitosan solution.

According to an example embodiment according to the present invention, Kit A is prepared by the same method as the method used for the preparation of the above radionuclide solution. A chitosan having a molecular weight of 460,000˜1,570,000 is dissolved in 1% acetic acid solution to prepare a chitosan solution having a viscosity of 380˜2,500 cps, and the chitosan solution is freeze-dried to prepare a freeze-dried chitosan to be used as Kit B. A radionuclide-chitosan complex solution having a viscosity of 300˜2,400 cps is prepared by mixing the radionuclide solution of Kit A and the freeze-dried chitosan of Kit B.

An appropriate viscosity of a radionuclide-chitosan complex solution according to the present invention is determined by observing viscosity changes for maximum 3 hours after the preparation of the complex solution, considering that a viscosity of the radionuclide-chitosan complex solution decreases as time passes and considering the time required for the treatment of a patient.

As a conclusion, the viscosity of the radionuclide-chitosan complex solution prepared by adding the radionuclide solution into the chitosan solution is preferably 300˜2,400 cps. The viscosity is more preferably 600˜2,400 cps considering that treatment for a patient is performed 2 or 3 hours after preparation of the radionuclide-chitosan complex solution. When the viscosity of the radionuclide-chitosan complex solution is lower than 300 cps, gels are dispersed due to an unstable gelation state. When the viscosity of the radionuclide-chitosan complex solution is higher than 2,400 cps, the viscosity of the radionuclide-chitosan complex solution is too high to be used for injection to a patient.

Additionally, a radionuclide-chitosan complex solution according to an example embodiment of the present invention may be prepared as follows.

1) A holmium-chitosan complex solution is prepared by mixing a freeze-dried chitosan (20 mg of chitosan/2 mL of 1% aqueous acetic acid solution) and 2 mL of holmium nitrate solution (containing 3.74 mg of holmium) to reduce chitosan and free holmium to be injected into the body and maintain a labeling yield above 99%.

In this case, the weight ratio of chitosan to holmium is 20 mg:3.74 mg (5.48:1 mole ratio) [200 mg of ¹⁶⁵Ho(NO₃)₃.5H₂O is dissolved in 2 mL of distilled water to give 10 wt. % of ¹⁶⁵Ho(NO₃)₃·5H₂O solution. 0.1 mL of ¹⁶⁵Ho(NO₃)₃.5H₂O solution contains 3.74 mg of holmium].

An appropriate mole ratio to combine chitosan with holmium is preferably 2˜30:1 (chitosan:holmium) [chitosan 1 mol:chitosan monomer 161 g, holmium 1 mol:holmium 165 g]. When the mole ratio of chitosan to holmium is less than 2 or is higher than 30, the quantity of free holmium increases. Through computer simulation and gelation experiments, it is identified that the mole ratio of chitosan to holmium is more preferably 3˜10:1, and the mole ratio is most preferably 3˜6:1.

2) A chitosan solution is freeze-dried for improvement of productivity during mass production, storage convenience, stability, and convenience in experiments and use. The freeze-dried chitosan is easily dissolved in a holmium solution and thereby a complex solution is easily prepared. The time required to prepare the complex solution by using a chitosan solution is about 1 to 2 hours. However, the required time can be reduced to 10 to 20 minutes by using a freeze-dried chitosan. Thus composition and method for preparing the holmium solution are properly modified because the freeze-dried chitosan is used instead of the chitosan solution.

3) A gelation state of a radionuclide-chitosan complex solution is observed that is prepared by using holmium-165 and a buffer solution having a pH similar to that inside the body. An appropriate viscosity of the radionuclide-chitosan complex solution is determined by checking viscosity changes through the observation of the gelation state. For experiments of viscosity changes according to the elapsed time, the radionuclide-chitosan complex solution is prepared by using holmium-166, which is a radioisotope, considering viscosity changes due to radiation.

The radionuclide-chitosan complex solution having a viscosity of 300˜2,400 cps may be directly injected into a lesion by a local injection. Arthritis and cystic cancers such as liver cancer, brain cancer, breast cancer and ovarian cancer may be treated by injecting the radionuclide-chitosan complex solution.

Accompanying drawings are briefly explained as follows.

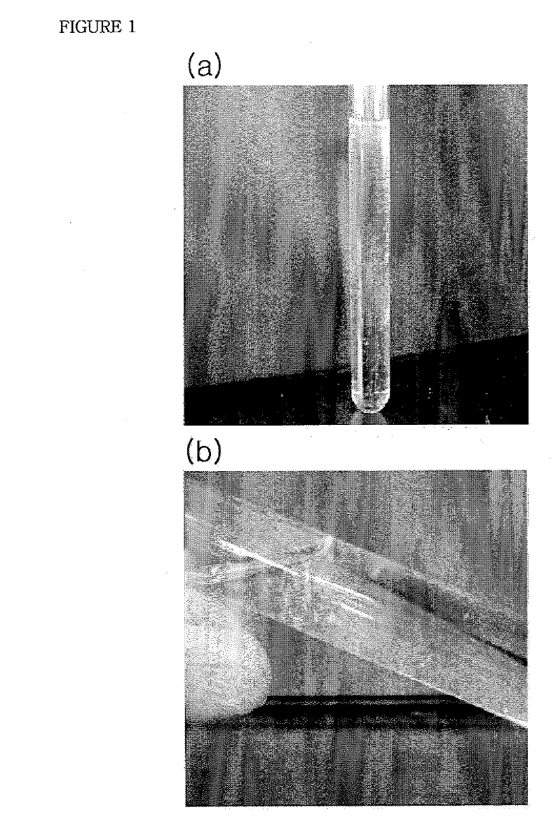

FIGS. 1a and 1b show gelation states of a holmium-chitosan complex solution having a viscosity is 60 cps and FIG. 1b shows gels dispersed.

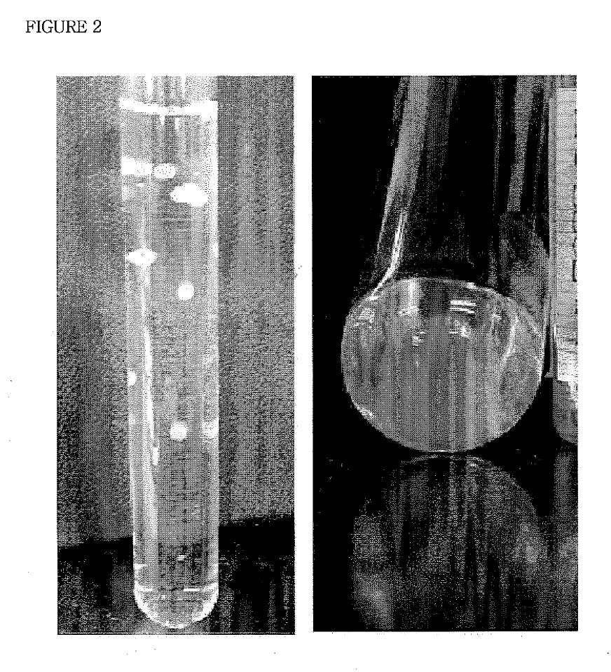

FIG. 2 shows a gelation state of a holmium-chitosan complex solution having a viscosity of 117 cps illustrating that gel droplets are formed, some of the gel droplets become dispersed and fall down (left picture), and some of the gel droplets are flocked together just before being dispersed (right picture).

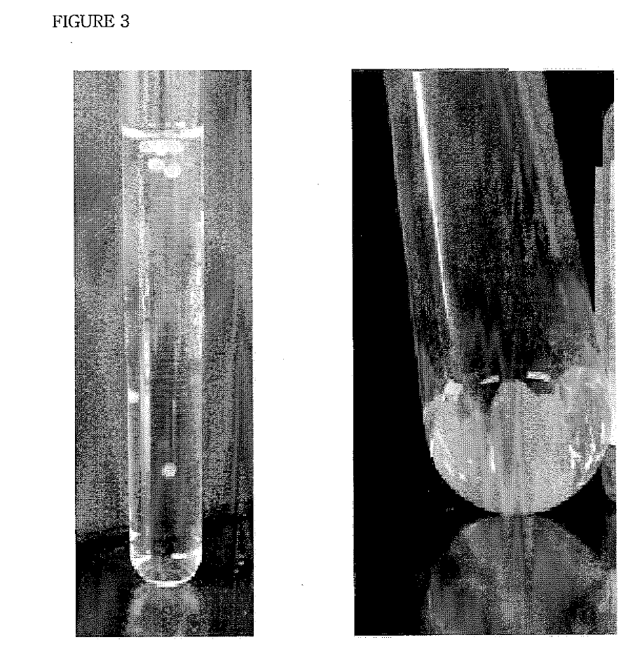

FIG. 3 shows a gelation state of a holmium-chitosan complex solution having a viscosity of 194 cps illustrating that gel droplets are formed, some of the gel droplets become dispersed and fall down (left picture) and some of the gel droplets are changed to a state to be easily dispersed (right picture).

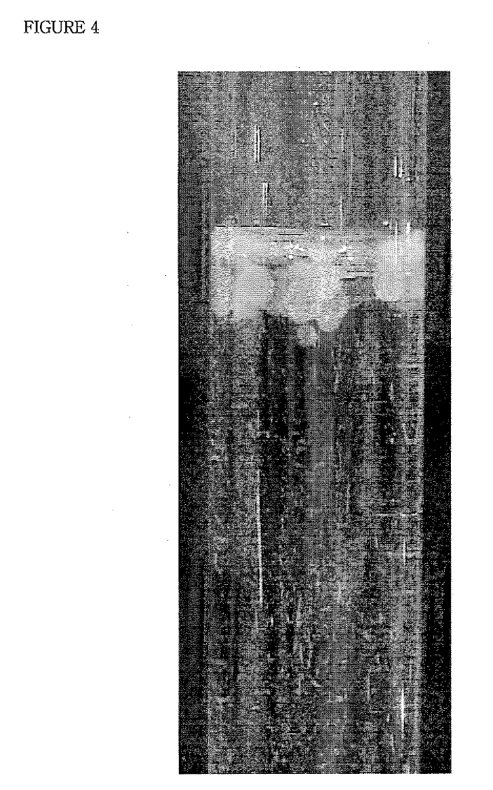

FIG. 4 shows a gelation state of a holmium-chitosan complex solution having a viscosity of 289 cps illustrating that gel droplets are formed but some of the gel droplets become dispersed.

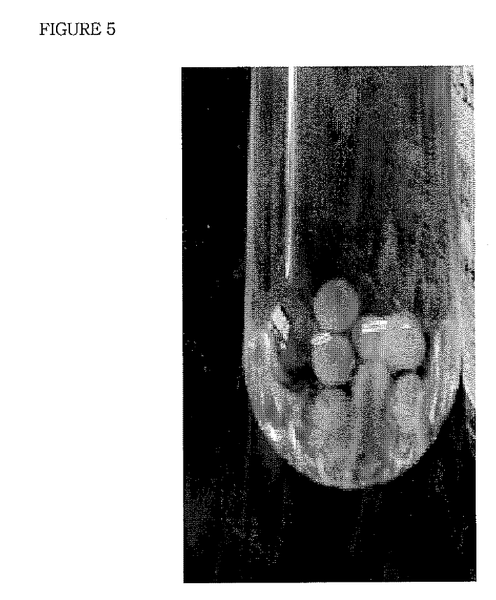

FIG. 5 shows a gelation state of a holmium-chitosan complex solution having a viscosity of 310 cps illustrating that gel droplets exist.

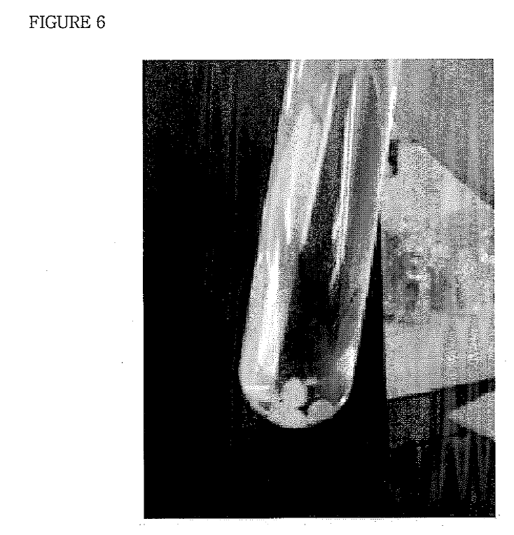

FIG. 6 shows a gelation state of a holmium-chitosan complex solution having a viscosity of 650 cps illustrating that gel droplets exist.

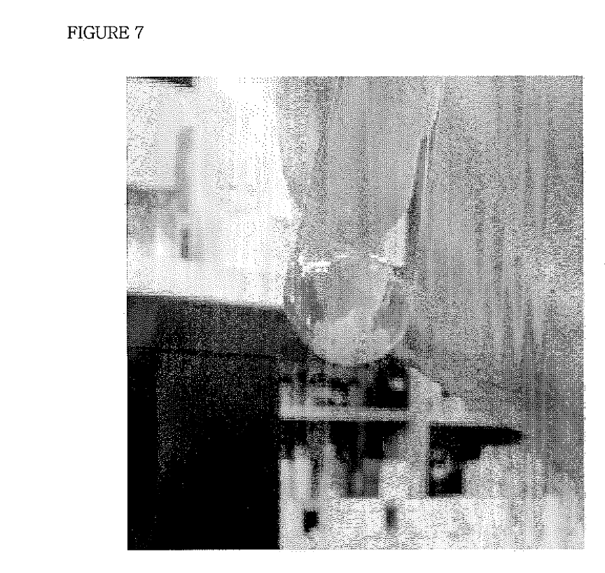

FIG. 7 shows a gelation state of a holmium-chitosan complex solution having a viscosity of 1,068 cps illustrating that gel droplets exist.

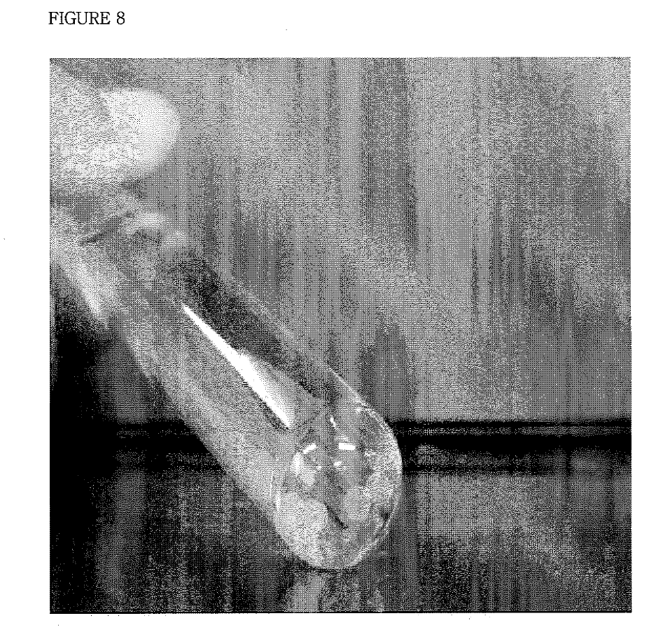

FIG. 8 shows a gelation state of a holmium-chitosan complex solution having a viscosity of 1,407 cps illustrating that gel droplets exist.

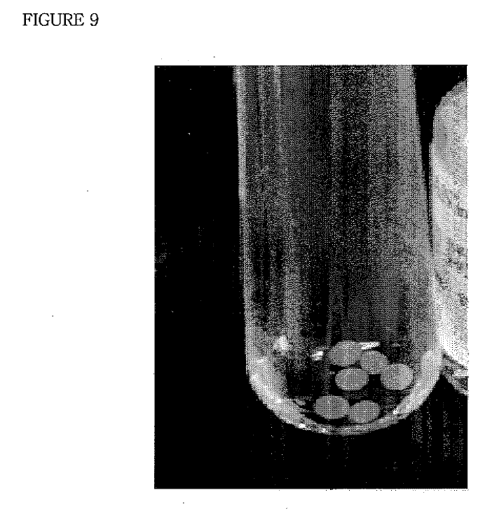

FIG. 9 shows a gelation state of a holmium-chitosan complex solution having a viscosity of 2,376 cps illustrating that gel droplets exist.

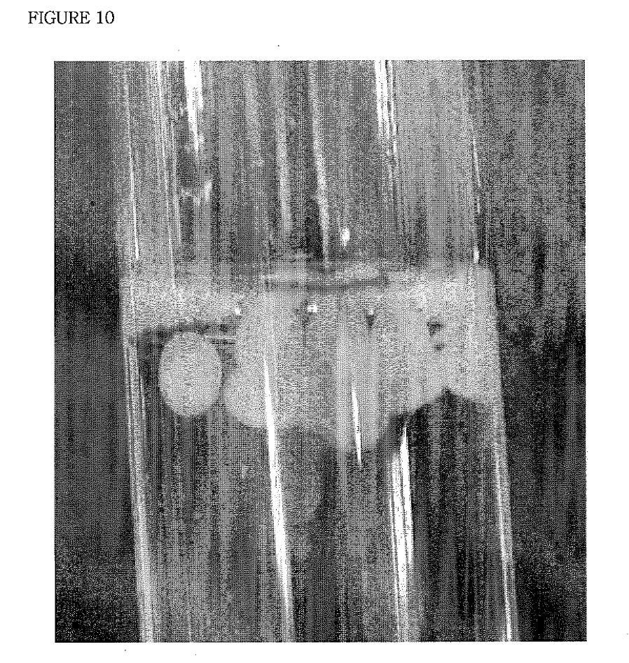

FIG. 10 shows a gelation state of a holmium-chitosan complex solution having a viscosity of 2,549 cps illustrating that gel droplets exist.

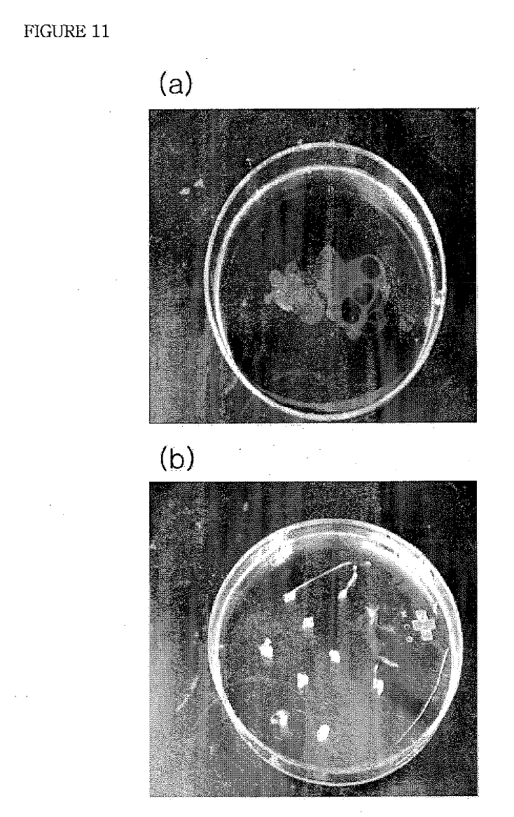

FIGS. 11a and 11b show a dispersed state and gelation state of holmium-chitosan complexes respectively. FIG. 11a shows that a complex solution is dispersed instead of gelation when the viscosity of the complex solution is less than 100 cps. FIG. 11b shows that gelation occurs at an injected region when the viscosity of a complex solution is more than 300 cps.

### MODE FOR INVENTION

Hereinafter, preferred example embodiments and experimental examples according to the present invention will be described in more detail. However, the present invention may be embodied in many different forms and should not be construed as limited to the example embodiments and experimental examples set forth herein.

### Examples 1˜5

### Preparation of Freeze-Dried Chitosans and Holmium-165 (A Stable Nuclide)-Chitosan Complex Solutions using Chitosans having Different Molecular Weights

To measure viscosities of holmium-165-chitosan complex solutions, chitosan solutions (20 mg of chitosan dissolved in 2 mL of 1% acetic acid) were prepared with chitosans having different molecular weights, were adjusted to pH 3.0 by 1NHCl solution and then viscosities are measured (summarized in Table 2). Each solution was freeze-dried to prepare a freeze-dried chitosan(Kit B). A holmium solution was prepared by using holmium nitrate [¹⁶⁵Ho(NO₃)₃.5H₂O] and 3.74 mg of holmium was dissolved in 2 mL of distilled water (Kit A). The Kit A was added into the Kit B with stirring. The viscosity of a mixture of the Kit A and B was measured after the mixture was stored for 30 minutes (summarized in Table 2).

Measurement of viscosity: Brookfield digital viscometer DV-II+

Measurement of molecular weight

1. System:
1) HPLC pump (Model: Waters 515)
2) Detector: Viscotek external RI detector
3) Column: Waters Ultrahydrogel™ 120

### Comparative Examples 1˜5

### Preparation of Freeze-Dried Chitosans and Holmium-165 (A Stable Nuclide)-Chitosan Complex Solutions using Chitosans having Different Molecular Weights

To measure viscosities of holmium-165-chitosan complex solutions, chitosan solutions (20 mg of chitosan dissolved in 2 mL of 1% acetic acid) were prepared with chitosans having different molecular weights, were adjusted to pH 3.0 by 1N HCl solution and then viscosities were measured (summarized in Table 2). Each solution is freeze-dried to prepare a freeze-dried chitosan (Kit B). A holmium solution was prepared by using holmium nitrate [¹⁶⁵Ho(NO₃)₃.5H₂O ] and 3.74 mg of holmium was dissolved in 2 mL of distilled water (Kit A). The Kit A was added into the Kit B with stirring. The viscosity of a mixture of the Kit A and B was measured after the mixture was stored for 30 minutes (summarized in Table 2).

TABLE 2
Viscosities (cps) of chitosan solutions and holmium-165 (a stable nuclide) - chitosan
complex solutions prepared using chitosans having different molecular weights

| | Comparative | Comparative | Comparative | Comparative | | | | | | Comparative |
|---|---|---|---|---|---|---|---|---|---|---|
| | example | example | example | example | Example | Example | Example | Example | Example | example |
| | 1 | 2 | 3 | 4 | 1 | 2 | 3 | 4 | 5 | 5 |
| Molecular | 62,500 | 112,400 | 173,900 | 419,500 | 462,000 | 874,900 | 1,098,000 | 1,325,000 | 1,569,000 | 1,672,000 |
| weight of | | | | | | | | | | |
| chitosan | | | | | | | | | | |
| Viscosity of | 71 | 148 | 255 | 351 | 382 | 688 | 1,173 | 1,544 | 2,489 | 2,610 |
| chitosan | | | | | | | | | | |
| solution | | | | | | | | | | |
| Viscosity of | 60 | 117 | 194 | 289 | 310 | 650 | 1,068 | 1,407 | 2,376 | 2,549 |
| complex | | | | | | | | | | |

| | | |
|---|---|---|
| Labeling | 50 | 99 |
| yield (%) | | |

### Experimental Example 1

### Gelation States of Holmium-165 (A Stable Nuclide)-Chitosan Complex Solutions Prepared using Chitosans having Different Molecular Weights (Examples 1˜5, Comparative Examples 1˜5)

Gelation states of holmium-165-chitosan complex solutions prepared according to Examples 1˜5 and Comparative examples 1˜5 were observed by taking the holmium-165-chitosan complex solutions into syringes and adding dropwise into buffer solutions of pH 7.02. The observed results were summarized in Table 3. The buffer solution was prepared according to USP listed method. The buffer solution was adjusted to be a concentration that was as same as the concentration showing osmotic pressure of human blood. The concentration of the buffer solution was 2 times the concentration of USP listed method.

The gelation states of Examples 1˜5 were stable and suitable for injection. The gelation states were stable when the viscosities of complex solutions were higher than 300 cps. Injection was not easy due to high viscosity when the viscosity of a complex solution was higher than 2,400 cps. Accordingly, the appropriate viscosity range of the complex solution for maintaining a stable gelation state in the body was 300˜2,400 cps. The molecular weight of the used chitosan was 460,000˜1,570,000 and the viscosity of the produced chitosan solution was 380˜2,500 cps.

TABLE 3
Gelation states of holmium-165 (a stable nuclide) - chitosan complex solutions prepared using
chitosans having different molecular weights (Examples 1~5, Comparative examples 1~5)

| Comparative | Comparative | Comparative | Comparative | | | | | | Comparative |
|---|---|---|---|---|---|---|---|---|---|
| example | example | example | example | Example | Example | Example | Example | Example | example |
| 1 | 2 | 3 | 4 | 1 | 2 | 3 | 4 | 5 | 5 |

| | | | | | |
|---|---|---|---|---|---|
| Gelation | Most | Gels are | Gels are | Gels are | Gels are formed as injected and are not dispersed |
| state | gels are | dispersed | partially | partially | |
| | dispersed | | dispersed | dispersed | |

| | | | |
|---|---|---|---|
| Remarks | Easy for injection | Easy for injection | Difficult for |
| | | | injection |

### Experimental Example 3

### Viscosity Changes of holmium-166 (A Radionuclide)-Chitosan Complex Solutions According to an Elapse of Time

A holmium-166-chitosan complex solution was prepared using ¹⁶⁶Ho(NO₃)₃.5H₂O instead of ¹⁶⁵Ho(NO₃)₃.5H₂O by the method used in the above examples. Viscosities of the holmium-166-chitosan complex solutions were measured according to an elapse of time and the results were summarized in Table 4 (viscometer:Brookfield digital viscometer DV-II+).

200 mg of ¹⁶⁵Ho(NO₃)₃.5H₂O stored in a polyethylene tube was irradiated with thermal neutrons having a velocity of 4.0×10¹³n/cm²·sec for 50 hours in an irradiation hole of a nuclear reactor (Hanaro at Korea Atomic Energy Research Institute) by using a pneumatic tube, and dissolved in water to produce the solution of ¹⁶⁶Ho(NO₃)₃.5H₂O.

It was identified that viscosity decreases as time passes. In order to maintain a complex's viscosity of 300 cps for about 3 hours, the viscosity of the complex solution (measured 30 minutes after mixing Kit A and Kit B) should preferably be 600 cps.

TABLE 4
Viscosity (cps) changes of holmium-166 (a radionuclide) -
chitosan complex solutions according to an elapse of time

| Elapsed time (hr) | 0.5 | 1 | 2 | 3 |
|---|---|---|---|---|
| Example 1′ | 305 | 279 | 242 | 183 |
| Example 2′ | 632 | 543 | 420 | 318 |
| Example 4′ | 1,367 | 1,223 | 1,055 | 874 |
| Example 5′ | 2,378 | 2,156 | 1,832 | 1,552 |
| Example 6 | 3,455 | 3,092 | 2,775 | 2,412 |

* The above Examples were prepared by using a radionuclide of holmium-166.

The holmium-chitosan complex solution as a medicine for direct injection into the body was prepared by mixing Kit A (holmium solution) and Kit B (freeze-dried chitosan) prior to use for treatment. In some cases, the complex solution was used 2 or 3 hours after preparation. Therefore, the viscosity of a complex solution should preferably be higher than 600 cps 30 minutes after preparation, considering that a viscosity of a complex solution decreases as the complex solution was hydrolyzed. Although the maximum viscosity of a complex solution might be 3455 cps theoretically, such a high viscosity of the complex solution gave difficulty in practical applications because radioactive holmium decayed as time passes, thereby exact radiation dose was difficult, influence due to radioactive decay should be considered during preparation and thereby preparation method was also complicated.

Therefore, the appropriate viscosity range of the radionuclide-chitosan complex solution was 300˜2,400 cps, and preferably 600˜2,400 cps considering that the treatment was performed about 3 hours after preparation of the complex solution.

### INDUSTRIAL APPLICABILITY

A radionuclide-chitosan complex solution according to the present invention has advantages that the labeling yield is above 99%, a gelation state is stably maintained at a target region when the radionuclide-chitosan complex solution is injected into the body and thereby radioactive materials are almost never leaked into other regions out of a target region. Side effects may be minimized and treatment efficiency is high when injected to a patient.

1-8. (canceled)

9. A radionuclide-chitosan complex solution having a viscosity of 300˜2,400 cps comprising a chitosan solution labeled with a radionuclide.

10. The radionuclide-chitosan complex solution of claim 9, wherein the chitosan solution is a chitosan solution having a viscosity of 380˜2,500 cps, which is prepared by dissolving a chitosan in a dilute acid solution.

11. The radionuclide-chitosan complex solution of claim 9, wherein the chitosan is a freeze-dried chitosan prepared by freeze-drying the chitosan solution of claim 10.

12. The radionuclide-chitosan complex solution of claim 9, wherein the radionuclide is ¹⁶⁶Ho.

13. A preparation method of a radionuclide-chitosan complex solution having a viscosity of 300˜2,400 cps, comprising:
(1) preparing a radionuclide solution by irradiating a stable nuclide selected from the group consisting of ¹⁶⁴Dy(NO₃)₃, ¹⁶⁴Dy₂O₃, ¹⁶⁵Ho(NO₃)₃ and ¹⁶⁵Ho₂O₃ with neutrons in a nuclear reactor to transform into a radionuclide and dissolving the radionuclide in distilled water,
(2) preparing a chitosan solution having a viscosity of 380˜2,500 cps by dissolving a chitosan in a weak acid solution, and
(3) adding the radionuclide solution prepared in step (1) into the chitosan solution prepared in step (2).

14. A preparation method of a radionuclide-chitosan complex solution having a viscosity of 300˜2,400 cps, comprising the steps of;
(1) preparing a radionuclide solution by irradiating a stable nuclide selected from the group consisting of ¹⁶⁴Dy(NO₃)₃, ¹⁶⁴Dy₂O₃, ¹⁶⁵Ho(NO₃)₃ and ¹⁶⁵Ho₂O₃ with neutrons in a nuclear reactor to transfonn into a radionuclide and dissolving the radionuclide in distilled water,
(2) preparing a freeze-dried chitosan by preparing a chitosan solution having a viscosity of 380˜2,500 cps through dissolution of a chitosan in a weak acid solution and freeze-drying, and
(3) adding the radionuclide solution prepared in step (1) into the freeze-dried chitosan prepared in step (2).

15. The preparation method of claim 13, wherein the mole ratio of the radionuclide to the chitosan is 1:2˜30.

16. The preparation method of claim 14, wherein the mole ratio of the radionuclide to the chitosan is 1:2˜30.

17. A method for treating liver cancer comprising administering a complex solution of claim 1 to a patient.
